(19) United States
(12) Patent Application Publication
(10) Pub. No.: US 2010/0222560 A1
(43) Pub. Date: Sep. 2, 2010

(54) UNIVERSAL COLUMN

(75) Inventor: Xiyu Jia, Newport Beach, CA (US)

Correspondence Address: SONNENSCHEIN NATH & ROSENTHAL LLP, P.O. BOX 061080, WACKER DRIVE STATION, WILLIS TOWER, CHICAGO, IL, 60606-1080, US

(73) Assignee: Zymo Research Corporation

(21) Appl. No.: 12/715,555

(22) Filed: Mar. 2, 2010

Related U.S. Application Data
(60) Provisional application No. 61/156,589, filed on Mar. 2, 2009.

(51) Int. Cl. C07H 21/04; B01D 15/08; C07H 21/00

(52) U.S. Cl. 536/23.1; 210/198.2; 536/25.41

(57) ABSTRACT

A universal column is provided which allows purification methods utilizing centrifugation, syringe coupling and/or use of a vacuum source. Methods for using the universal column and kits comprising the universal column are described.

This application claims priority to U.S. Provisional Application No. 61/156,589, filed on Mar. 2, 2009 which is incorporated herein by reference in its entirety.

### BACKGROUND

Chromatography is used for the purification of a wide range of compounds. There remains a need, however, for improved chromatography columns that incorporate features to provide versatility and improved efficiency. The instant disclosure provides a new highly versatile universal column and improved purification methods that address this need.

### SUMMARY

In one illustrative embodiment, the column assembly includes a body having an upper section, a reservoir section, and a lower section. A reservoir is formed in the body adjacent the reservoir section and is arranged to store a binding matrix. A top coupling member is disposed adjacent the upper section, and the top coupling member has a top passage in flow communication with the reservoir. The reservoir section may or may not comprise a void. For example, a portion of, or the entire, reservoir section may be comprised of a binding matrix or other porous material that facilitates flow communication. The top coupling member is configured to couple to a syringe or a reservoir adapter. An inner projection is formed adjacent the lower section, the inner projection having a bottom passage in flow communication with the reservoir, the inner projection being sized to connect to a vacuum manifold. An outer projection surrounds a portion of the inner projection, and the outer projection is sized to engage a centrifuge tube (e.g., a 1.5 to 2.0 ml microcentrifuge tube). Therefore, the column assembly can be coupled to a syringe, a reservoir adapter, a vacuum manifold, or a centrifuge tube to enable fluid to pass through the binding matrix.

In a further embodiment, the reservoir section, the inner projection, and the outer projection are cylindrical. An outer diameter of the outer projection may be smaller than an outer diameter of the reservoir section. Specifically, an outer diameter of the outer projection may be between about 4 mm and 11 mm. For example, outer diameter of the outer projection may be about 4, 5, 6, 7, 8, 9, 10 or 11 mm.

In a still further embodiment, a bottom portion of the inner projection may axially extend beyond a bottom portion of the outer projection. In certain aspects, the outer projection may extend between about 4 mm to about 35 mm from the reservoir and the inner projection may extend between about 6 mm to about 35 mm from the reservoir. For example, the outer projection may extend about 14 mm from the reservoir and the inner projection may extend about 18 mm from the reservoir. In one more embodiment, an inner projection and the outer projection may be integrally formed.

In another embodiment, the column assembly may also include a support structure within the reservoir arranged to support the binding matrix. Specifically, the support structure may comprise a plurality of support ribs, the support ribs being integrally formed with a bottom portion of the reservoir.

In a further embodiment, the top coupling member may include at least two mating tabs extending radially from a top portion of the top coupling member.

In one more embodiment, the body may be formed from a thermoplastic polymer. Moreover, the thermoplastic polymer may be formed from polypropylene, polystyrene, or a mixture of polypropylene and polystyrene.

In yet another embodiment, the top coupling member may be ultrasonically welded to a top portion of the reservoir section. The skilled artisan will recognize, however, that the top coupling member may be coupled the top portion of the reservoir by other mechanisms, such as by screwing, gluing or snapping the members together.

A further embodiment of the column assembly includes a body having an upper section, an intermediate section, a reservoir section, and a lower section. A reservoir is formed within the reservoir section and arranged to store a binding matrix. The reservoir section is sized to engage a centrifuge tube (e.g., a 1.5 to 2.0 ml microcentrifuge tube). A collar is disposed adjacent the intermediate section, and the collar has an interior portion in flow communication with the reservoir. A top coupling member is disposed adjacent the upper section, and the top coupling member has a top passage in flow communication with the interior portion of the collar. The top coupling member is configured to couple to a syringe or a reservoir adapter. Additionally, a bottom coupling member is disposed adjacent the lower section, the bottom coupling member having a bottom passage in flow communication with the reservoir. The bottom coupling member is sized connect to a vacuum manifold. Therefore, the column assembly can be coupled to a syringe, a reservoir adapter, a vacuum manifold, or a centrifuge tube to enable fluid to pass through the binding matrix.

In a still further embodiment, the reservoir section is cylindrical. The collar may also be cylindrical, and an outer diameter of the reservoir section may be smaller than an outer diameter of the collar. The bottom coupling member may also be cylindrical, and an outer diameter of the bottom coupling member may be smaller than an outer diameter of the reservoir section. The outer diameter of the reservoir section may be between about 4 mm and 11 mm. For example, the outer diameter of the reservoir section may be may be about 4, 5, 6, 7, 8, 9, 10 or 11 mm. In certain further aspects, the reservoir section may have a length of between about 4 mm and about 35 mm (e.g., about 14 mm).

In one more embodiment, the collar, the reservoir section, and the bottom coupling member may be integrally formed.

In another embodiment, the column assembly may also include a support structure within the reservoir arranged to support the binding matrix. Specifically, the support structure may comprise a plurality of support ribs, the support ribs being integrally formed with a bottom portion of the reservoir.

In a still further embodiment, the top coupling member may include at least two mating tabs extending radially from a top portion of the top coupling member.

In a further embodiment, the body may be formed from a thermoplastic polymer. Moreover, the thermoplastic polymer may be formed from polypropylene, polystyrene, or a mixture of polypropylene and polystyrene.

In another embodiment, the top coupling member may be ultrasonically welded to a top portion of the reservoir section. Alternatively or additionally, the top coupling member may be coupled the top portion of the reservoir by other mechanisms, such as by screwing, gluing or snapping the members together.

In one more embodiment, the reservoir section may include a tapered portion integrally formed with a bottom portion of the reservoir section and a top portion of the bottom coupling member, the tapered portion having an interior portion that is in flow communication with the bottom passage.

In a further embodiment there is provided a method for separating a compound from impurities comprising: (i) loading a sample that comprises a compound and impurities onto a column as described herein (e.g., see FIGS. 1 and 2) wherein the column comprises a binding matrix; (ii) incubating the column under conditions wherein the compound binds to the column matrix; (iii) removing impurities from the column under conditions wherein the compound remains bound to the column matrix.

In certain aspects, the loading (i) and incubating (ii) steps of the method are performed simultaneously under conditions wherein the compound binds to the matrix. Moreover, in some cases, the loading (i), incubating (ii) and removing impurities (ii) steps may be performed simultaneously. For example, sample comprising a compound and impurities may be passed through a column under conditions wherein the compound binds to the column matrix and one or more impurities flow through the column.

In a further embodiment, a method for separating a compound from impurities further comprises (iv) removing the compound from the column eluting the compound from the column to provide a purified compound. For example, the removing step (iv), in some aspects, comprises applying an elution buffer to the column under conditions in which the compound is released from the matrix and collecting the elution buffer comprising the compound. Moreover, in some embodiments, the step of removing impurities from the column (iii), further comprises washing the column matrix one or more times with a wash buffer wherein the compound remains bound to the column matrix.

Alternatively or additionally, there is provided a method for separating a compound from impurities comprising: (i) loading a sample that comprises a compound and impurities onto a column as described herein wherein the column comprises a binding matrix; (ii) incubating the column under conditions wherein one or more impurities binds to the column matrix; (iii) removing compound from the column under conditions wherein one or more impurities remain bound to the column matrix.

A used herein a “compound” refers to a molecule or a complex of molecules. For example, a compound may be a protein, a protein complex, a carbohydrate, a nucleic acid, a lipid or a complex thereof such as a cell or virus. In certain aspects, the compound is a nucleic acid such as a DNA (e.g., plasmid DNA) or a RNA molecule. For example, methods for purifying nucleic acids which may used in the context of the current disclosure are described in U.S. Publication No. 20070015169, incorporated herein by reference.

As used herein a “sample” refers to a solution or suspension that comprises a compound (e.g., an aqueous solution). For example, the sample is, in certain aspects, a body fluid (e.g., a blood, saliva or urine sample), a cell preparation or a cell lysate. It is contemplated that cell lysates may be from eukaryotic cells, such as mammalian cells or from prokaryotic cells such as gram-negative bacteria (e.g., E. coli).

In certain aspects, methods disclosed herein concern loading a sample (comprising impurities), a wash buffer or an elution buffer onto a column and then removing said buffer or impurities. The skilled artisan will recognize that gravity may be used to allow a solution applied to a column to pass through the column. However, in some cases, a force is applied to move the solution though the column. For example, a positive pressure can be applied to the top of a column. One example of a procedure to apply a positive pressure to the top of a column is “push” a solution or suspension through a column by depressing the plunger of a syringe connected to the column. In another example, a negative pressure can be applied to the bottom of a column to move a solution through the column. For instance, vacuum source (e.g., a vacuum manifold or a syringe, wherein the plunger is pulled back) can be applied to “pull” a solution through the column. Alternatively or additionally, a column may be spun in centrifuge to move a solution through the column. Thus, in some cases, removing the sample or one or more impurities in step (iii) comprises at least one procedure selected from the group consisting of spinning the column in centrifuge, applying a positive pressure to the top of the column and applying a negative pressure to the bottom of the column. Likewise, a wash buffer is, in certain aspects, can be removed by spinning the column in centrifuge, by applying a positive pressure to the top of the column or by applying a negative pressure to the bottom of the column. Moreover, collecting the elution buffer which comprises the compound can comprise spinning the column in centrifuge, applying a positive pressure to the top of the column or applying a negative pressure to the bottom of the column (e.g., collecting the elution buffer and compound in a syringe). Thus, the skilled worker will recognize that, in certain aspects, a purification method according to the instant disclosure may employ only a syringe to move solutions through the column. In certain cases, methods disclosed herein do not employ a procedure involving a centrifuge or a centrifugation step.

In a further embodiment, there is provided a purification kit comprising a universal column as described herein and one or more additional components selected from the group consisting of: a preparative buffer, an elution buffer, a wash buffer, a reservoir adapter (e.g., a reservoir adapter comprises a filter), a syringe, a centrifuge tube, a microcentrifuge tube, a collection tube, a nuclease, and an instruction manual for use of the kit. For example, the preparative buffer may be a cell lysis buffer or neutralization buffer. In certain aspects, kit components may be packaged together in a box or crate. In some cases, a kit comprises a plurality of columns such as 10, 15, 20, 25, 30, 35, 40, 45, 50 or more columns packaged together with other elements of the kit.

Other features and advantages of the invention will be better understood by reference to the detailed description of illustrative embodiments that follow.

### BRIEF DESCRIPTION OF THE DRAWING

The following drawings are part of the present specification and are included to further demonstrate certain aspects of the present invention. The invention may be better understood by reference to the drawings in combination with the detailed description of specific embodiments presented herein.

FIG. 1A: A front view of a column assembly of the present disclosure.

FIG. 1B: A top view of the column assembly of FIG. 1A.

FIG. 1C: A longitudinal cross-sectional view of the column assembly of FIGS. 1A and 1B taken along line 1C-1C in FIG. 1B.

FIG. 2A: A front view of another illustrative embodiment of a column assembly of the present disclosure.

FIG. 2B: A top view of the column assembly of FIG. 2A.

FIG. 2C: A longitudinal cross-sectional view of the column assembly of FIGS. 2A and 2B taken along line 2C-2C of FIG. 2B.

FIG. 3A: A front view of an illustrative embodiment of a column assembly of the present disclosure coupled to a syringe;

FIG. 3B: A front view of an illustrative embodiment of a column assembly of the present disclosure coupled to a vacuum manifold;

FIG. 3C: A front view of an illustrative embodiment of a column assembly of the present disclosure coupled to a reservoir adapter;

FIG. 3D: A front view of an illustrative embodiment of a column assembly of the present disclosure coupled to a reservoir adapter, wherein the reservoir adapter is secured inside a tube;

FIG. 3E: A front view of an illustrative embodiment of a column assembly of the present disclosure coupled to a microcentrifuge tube which is shown in cross section.

FIG. 3F: A front view of an illustrative embodiment of a column assembly of the present disclosure coupled to a microcentrifuge tube;

FIG. 4A: A front view of an illustrative embodiment of a column assembly of the present disclosure; and

FIG. 4B: A lateral cross-sectional view of the column assembly of FIG. 4A.

### DETAILED DESCRIPTION

The following is a detailed description of the invention provided to aid those skilled in the art in practicing the present invention. Those of ordinary skill in the art may make modifications and variations in the embodiments described herein without departing from the spirit or scope of the present invention.

### The Column Assembly

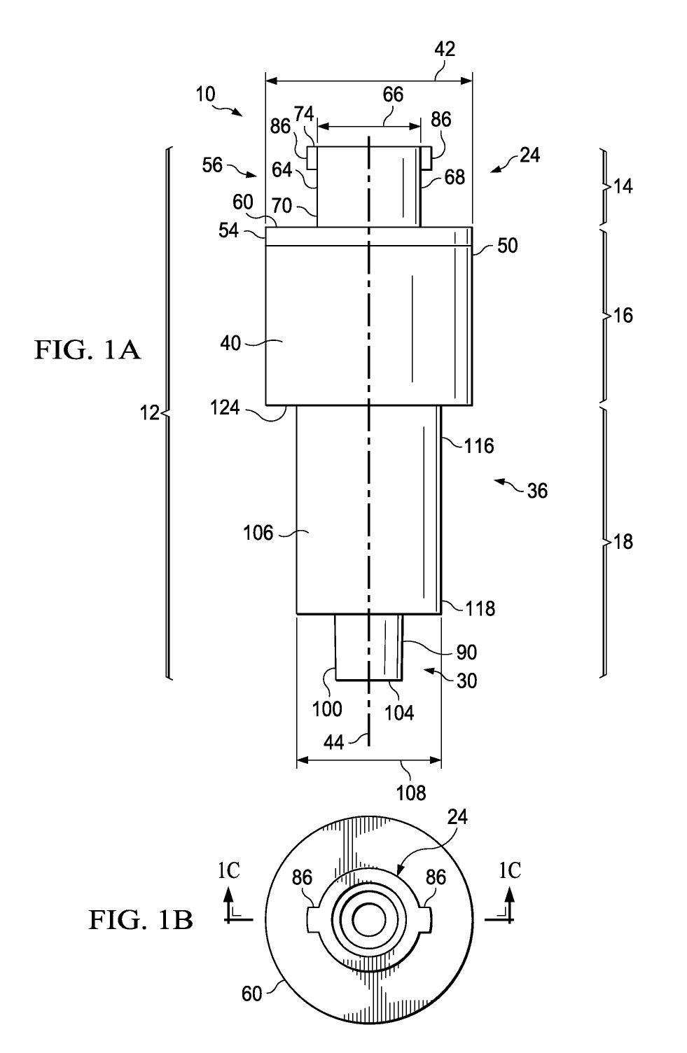

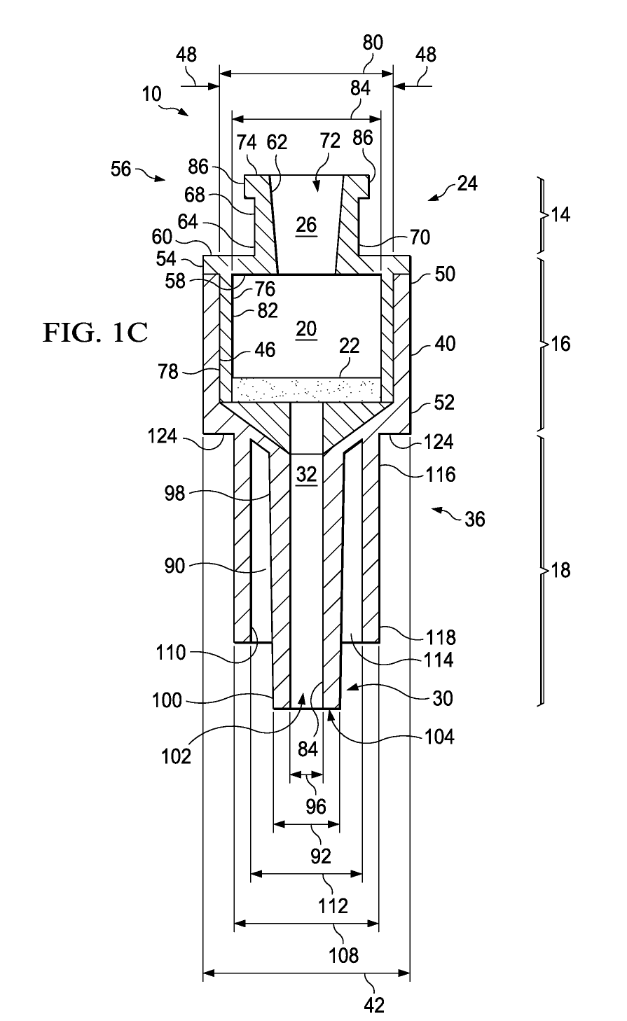

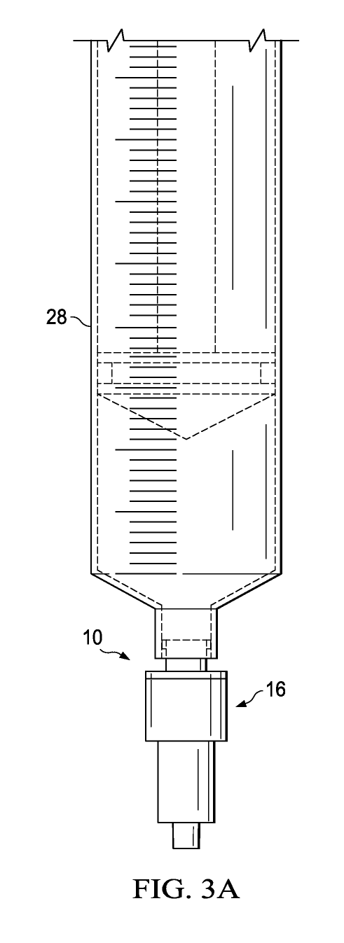

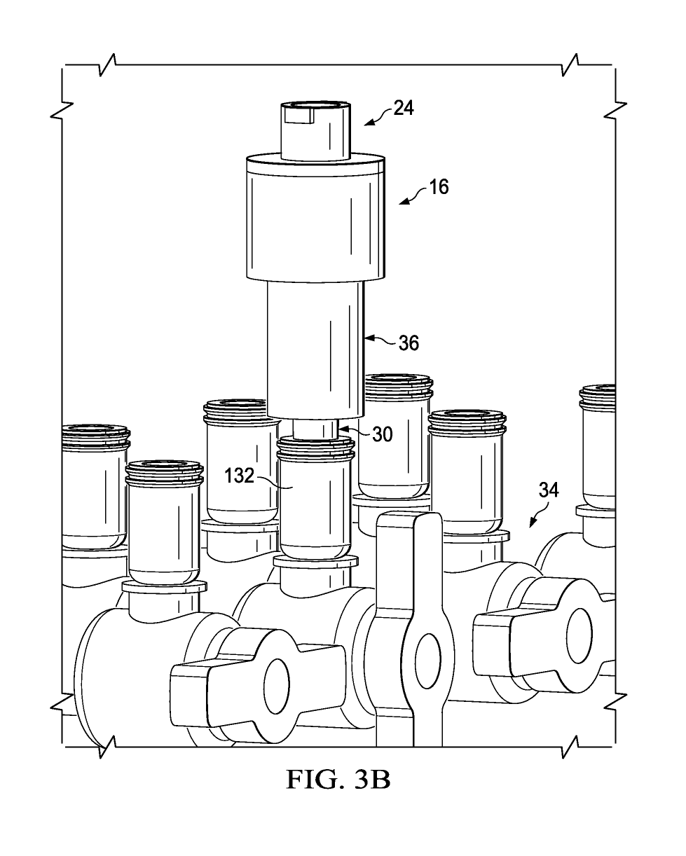

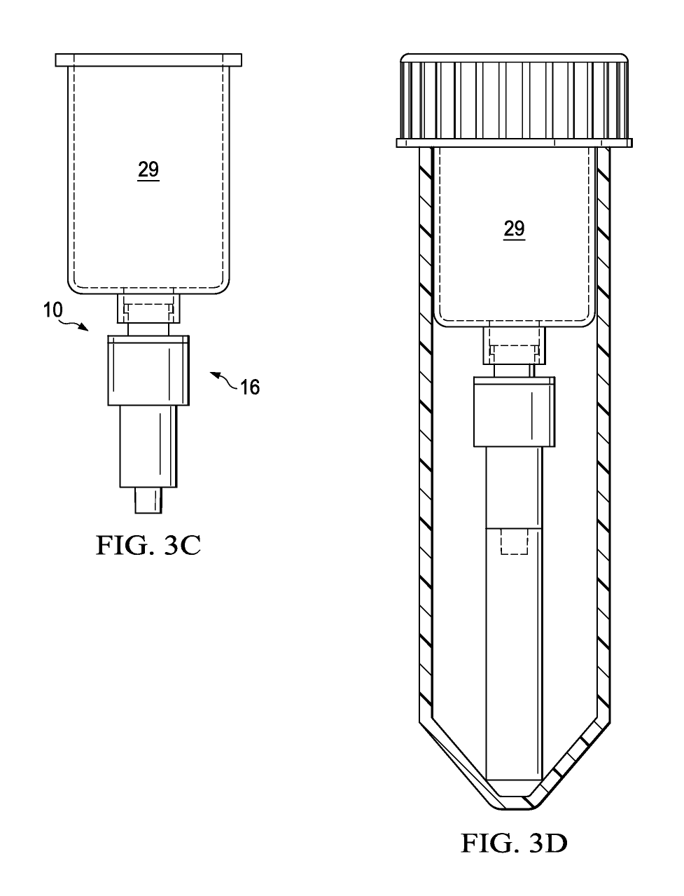

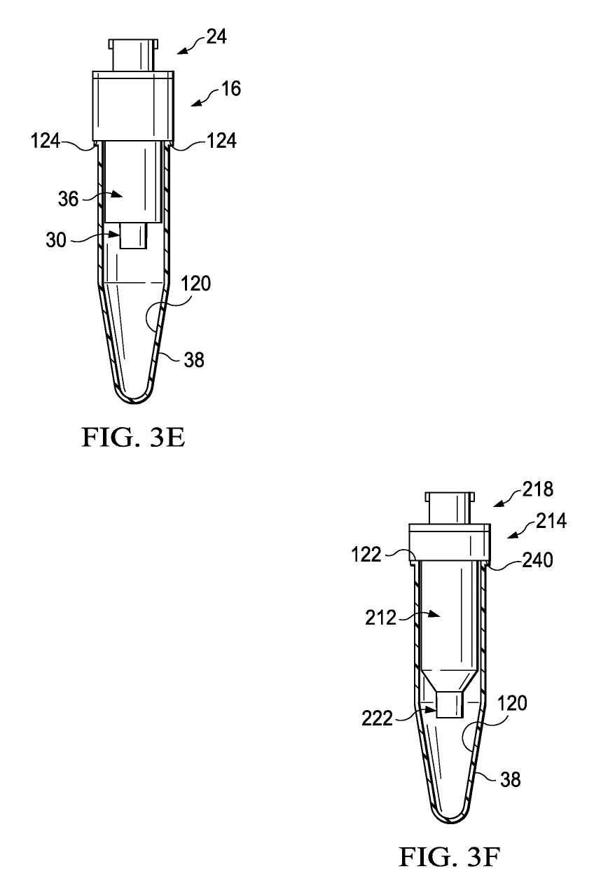

Referring to FIGS. 1A-1C, an illustrative embodiment of a column assembly 10 includes a body 12 having an upper section 14, a reservoir section 16, and a lower section 18. A reservoir 20 is formed in the body 12 adjacent the reservoir section 16 and is arranged to store a binding matrix 22. A top coupling member 24 is disposed adjacent the upper section 14, and the top coupling member 24 has a top passage 26 in flow communication with the reservoir 20. The top coupling member 24 is configured to couple to a syringe 28 (FIG. 3A) or a reservoir adapter 29 (FIG. 3C). An inner projection 30 is formed adjacent the lower section 18. The inner projection 30 has a bottom passage 32 in flow communication with the reservoir 20, and the inner projection 30 sized and configured to connect to a vacuum manifold 34 (FIG. 3B). An outer projection 36 surrounds a portion of the inner projection 30, and the outer projection 36 is sized and configured to engage a centrifuge tube 38 (FIG. 3E). The body 12 is preferably formed from a plastic material. For example, the body 12 may be formed from a thermoplastic polymer, such as polypropylene, polystyrene, or a mixture of polypropylene or polystyrene. Other materials may also prove suitable.

The reservoir section 16 of the body 12 of column assembly 10 includes the reservoir 20. The reservoir section 16 may have a circular cross-sectional shape such that an exterior surface 40 of the reservoir section 16 has a cylindrical shape having an outer diameter 42 perpendicular to a longitudinal axis 44 of the reservoir section 16. However, the reservoir section 16 may have any suitable cross-sectional shape, including, for example, a polygon or an oval. The reservoir section 16 may also have an interior surface 46 at least partially defining the reservoir 20 within the reservoir section 16, and the interior surface 46 may have an inner diameter 48 perpendicular to the longitudinal axis 44. Additionally, the reservoir section 16 may have a top portion 50 and a bottom portion 52 longitudinally opposite the top portion 50. As used herein, the term “diameter” is used to describe both the actual diameter of a designated element having a circular cross-sectional shape and also the diameter of a circle generally circumscribing as much of a cross-sectional perimeter of an element having a non-circular cross-sectional shape as possible.

Referring still to FIGS. 1A-1C, the upper section 14 of the body 12 of column assembly 10 additionally includes the top coupling member 24. The top coupling member 24 may include a base portion 54 and a top projection 56 extending away from the base portion 54. The base portion 54 may have the general shape of a disk having an outer diameter approximately equal to the outer diameter 42 of the reservoir section 16. The base portion 54 may have a flange, or planar bottom surface 58, configured to mate with the top portion 50 of the reservoir section 16 and a planar top surface 60 opposite the bottom surface 58. The top projection 18 may extend from the top surface 60 of the base portion 54 in the direction of the longitudinal axis 44 of the reservoir section 16. The top projection 56 may have an interior surface 62 and an exterior surface 64 having an outer diameter 66 perpendicular to the longitudinal axis 44. The top projection 56 may also have a top portion 68 and a bottom portion 70, the bottom portion 70 being proximate to the top surface 60 of the base portion 54. An aperture 72 may be disposed on a top surface 74 of the top projection 56, and the aperture 72 and the interior surface 62 of the top projection 56 may form the top passage 26. The top passage 26 may extend longitudinally through the top coupling member 24 such that, when the top coupling member 24 is secured to the reservoir section 16, the top passage 26 is in flow communication with the reservoir 20.

Referring primarily now to FIG. 1C, the top coupling member 24 may include a reservoir projection 76 to assist with centering the top coupling member 24 to the reservoir section 16 during assembly. The reservoir projection 76 may extend away from the base portion 54 in a direction opposite the top projection 56 such that the reservoir projection 76 is received into the reservoir 20. Accordingly, an exterior surface 78 of the reservoir projection 76 may have an outer diameter 80 sized to be slightly smaller than the inner diameter 48 of the reservoir section 16 such that the exterior surface 78 mates with the interior surface 46 of the reservoir section 16. The reservoir projection 76 may also include an interior surface 82, the interior surface 82 having an inner diameter 84 that is dimensioned such that fluid flow through the top passage 26 is not obstructed by the reservoir projection 76. The top coupling member 24 may be secured to the reservoir section 16 by any method known in the art, such as ultrasonic welding, adhesive bonding, interference fitting, other device, or any combination thereof. For instance, the bottom surface 58 of the base portion 54 may be ultrasonically welded to the top portion 50 of the reservoir section 16. Preferably, a fluid-permeable binding matrix 22 having a diameter approximately equal to the inner diameter 48 of the reservoir section 16 is inserted or formed into the reservoir 20 prior to securing the top coupling member 24 to the reservoir section 16.

The top coupling member 24 may also include a pair of mating tabs 86 that extend radially from the top portion 68 of the top projection 56, as shown in FIGS. 1A-1C. The mating tabs 86 are configured to be compatible with a mating connection, such as a Luer-Lok® and/or LuerSlip® mating system commonly used to provide leak-free connections between medical or laboratory instruments. Preferably, the mating tabs 86 may be used to couple the top coupling member 24 to a syringe 28, as illustrated in FIG. 3A, or to a hollow, cylindrical reservoir adapter 29, as illustrated in FIG. 3C.

Referring again to FIGS. 1A and 1C, the lower section 18 of the body 12 of column assembly 10 includes an inner projection 30 and an outer projection 36 coupled to the bottom portion 52 of the reservoir section 16. The lower section 18 may be integrally formed with the reservoir section 16, or may be secured to the reservoir section 16 by any method used in the art, such as ultrasonic welding, adhesive bonding, or interference fitting. The inner projection 30 and the outer projection 36 may both extend longitudinally away from the reservoir section 16 in the same direction as the longitudinal axis 44 of the reservoir section 16. The inner projection 30 may have a circular cross-sectional shape such that an exterior surface 90 of the inner projection 30 has a cylindrical shape having an outer diameter 92 when perpendicular to the longitudinal axis 44 of the reservoir section 16. However, the inner projection 30 may have any suitable cross-sectional shape, including, for example, a polygon or an oval. The inner projection 30 may also include an interior surface 84 having an inner diameter 96, as well as a top portion 98 and a bottom portion 100. An aperture 102 formed on a bottom surface 104 of the inner projection 30 and the interior surface 94 may define a bottom passage 32 extending through the lower section 18 such that the bottom passage 32 is in flow communication with the reservoir 20.

The outer projection 36 of the lower section 18 may be secured to or integrally formed with the reservoir section 16 and may extend longitudinally in the same direction as the longitudinal axis 44 of the reservoir section 16. The outer projection 36 may also include an exterior surface 106 having an outer diameter 108 and an interior surface 110 having an inner diameter 112 such that a gap 114 is formed between the interior surface 110 and the exterior surface 90 of the inner projection 30, as shown in FIG. 1C. Conversely, the outer projection 36 may be integrally formed with the exterior surface 90 of the inner projection 30 such that there is no gap between the between the interior surface 110 and the exterior surface 90 of the inner projection 30. The outer projection 36 may also include a top portion 116 and a bottom portion 118 longitudinally opposite the top portion 116. As illustrated in FIGS. 1C and 3E, the outer diameter 108 of the outer projection 36 is smaller than the outer diameter 42 of the reservoir section 16 and is dimensioned to be received into a standard microcentrifuge tube 38, such as an Eppendorf® tube, such that the exterior surface 106 of the outer projection 36 mates with an interior surface 120 within the microcentrifuge tube 38, and a top surface 122 of the microcentrifuge tube 38 mates with, or abuts to, a reservoir shoulder 124 formed by the bottom portion 52 of the reservoir section 16 extending past the exterior surface 106 of the outer projection 36. In one embodiment, the outer diameter 108 of the outer projection 36 may be between about 4 mm and 11 mm (e.g., about 8.9 mm), and the outer diameter 42 of the reservoir section 16 may be approximately 12.5 mm to accommodate a standard microcentrifuge tube 38. Moreover, the bottom portion 100 of inner projection 30 should project beyond the bottom portion 118 of the outer projection 36 to allow the coupling of a vacuum manifold 34 (FIG. 3B) to be secured to the bottom portion 100 of the inner projection 30 without obstruction by the bottom portion 118 of the outer projection 36. In one embodiment, the distance between the bottom portion 100 of the inner projection 30 and the bottom portion 118 of the outer projection 36 is approximately 4 mm.

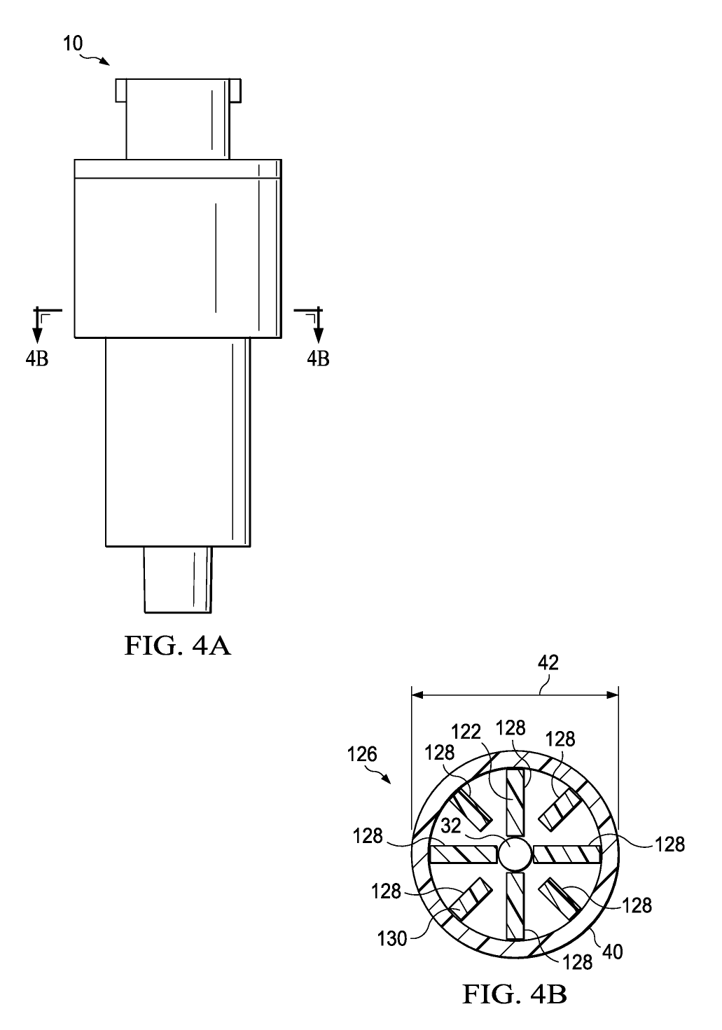

Referring to FIGS. 1C, 4A, and 4B, the reservoir structure 16 of the body 12 of column assembly 10 may also include a support structure 126 within the reservoir 20 arranged to support the binding matrix 22. The support structure 126 may include a plurality of support ribs 128 disposed within the reservoir 20 proximate to the bottom portion 52. Each of the plurality of support ribs 128 may be integrally formed with the reservoir section 16. Each of the plurality of support ribs 128 may have a planar top surface 130 that is approximately normal to the longitudinal axis 44 of the reservoir section 16, the plurality of top surfaces 130 being configured to support the binding matrix 22 within the reservoir 20. The plurality of support ribs 128 may form a symmetrical array around the bottom passage 32 when viewed along the longitudinal axis 44 of the reservoir section 16 such that the bottom passage 32 is not obstructed. In one embodiment, the support ribs may be arrayed in 45 degree intervals.

When it is desired to draw a fluid through the binding matrix 22 supported within the reservoir 20 of the column assembly 10, several methods can be employed. First, a syringe 28 containing a fluid can be coupled to the top projection 56 of the top coupling member 24 using a mating connection, e.g., Luer-Lok® coupling mechanism described above and shown in FIG. 3A. The column assembly 10 may also be coupled to a reservoir adapter 29 in the same manner, as illustrated in FIG. 3C. Second, the column assembly 10 may be coupled to a vacuum manifold 34 by inserting a stopcock 132 into the bottom passage 32 of the inner projection 30, as illustrated in FIG. 3B. However, an adapter, such as a tube (not shown), may be used to couple the stopcock 132 to the inner projection 30. Finally, the column assembly 10 can be coupled to a microcentrifuge tube 38 as previously described and as shown in FIG. 3E, and the microcentrifuge tube 38 can then be inserted into a centrifuge (not shown).

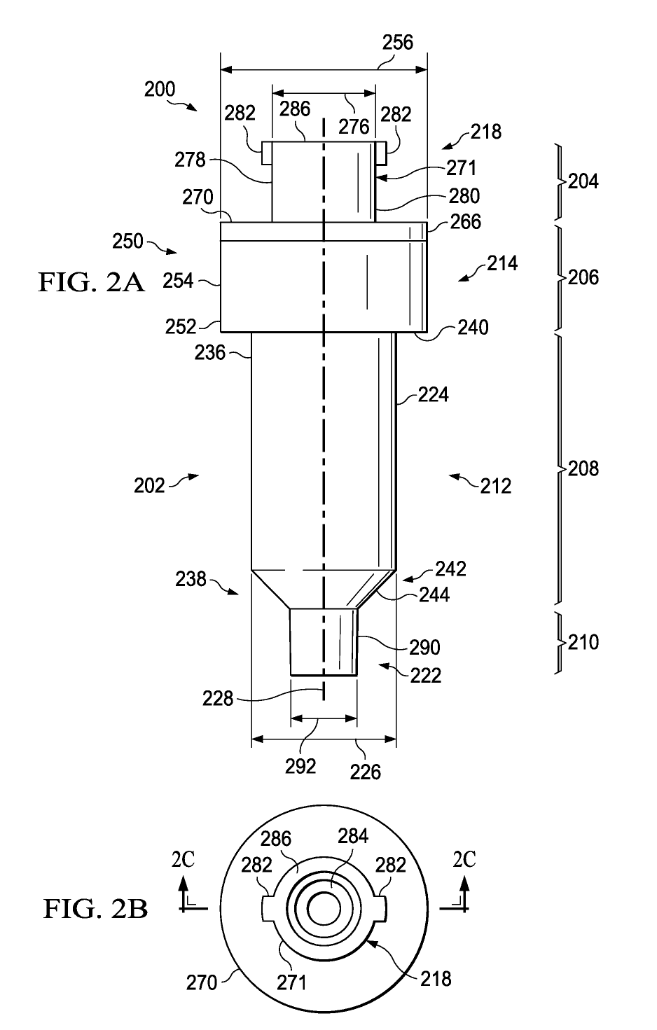

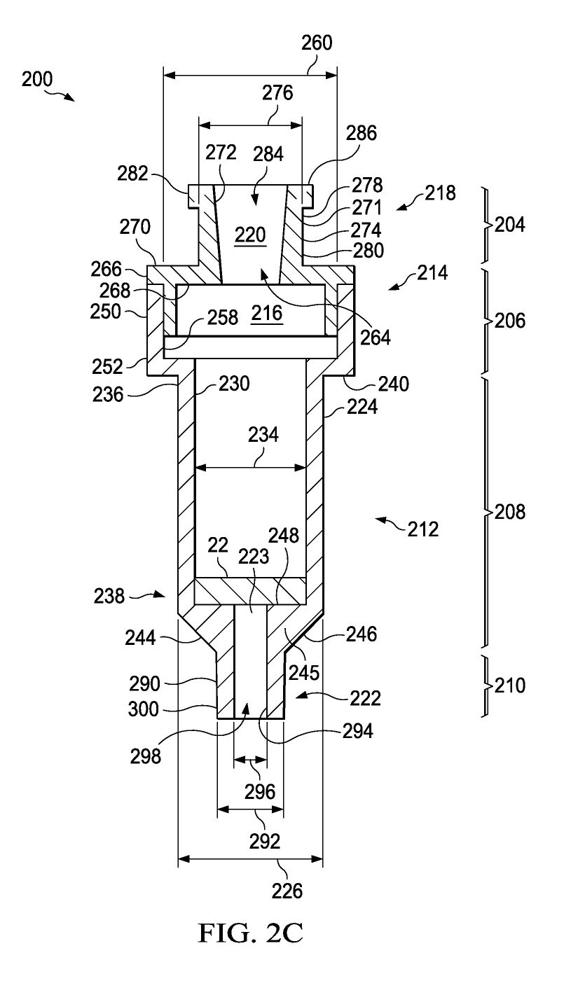

Referring now primarily to FIG. 2A, a second embodiment of a column assembly 200 includes a body 202 having an upper section 204, an intermediate section 206, a reservoir section 208, and a lower section 210. A reservoir 212 is formed within the reservoir section 208 and arranged to store a binding matrix 22. The reservoir section 208 is sized and configured to engage a centrifuge tube 38 (FIG. 3E). A collar 214 is disposed adjacent the intermediate section 206, and the collar 214 has an interior portion 216 in flow communication with the reservoir 212. A top coupling member 218 is disposed adjacent the upper section 204, the top coupling member 218 having a top passage 220 in flow communication with the interior portion 216 of the collar 214. The top coupling member 218 is configured to couple to a syringe 28 or a reservoir adapter 29. Additionally, a bottom coupling member 222 is disposed adjacent the lower section 210, the bottom coupling member 222 having a bottom passage 223 in flow communication with the reservoir 212. The bottom coupling member 222 is sized and configured to connect to a vacuum manifold 34. The body 202 may be formed from a plastic material. For example, the body 202 may be formed from a thermoplastic polymer, such as polypropylene, polystyrene, or a mixture of polypropylene or polystyrene. Other materials may also prove suitable.

As illustrated in FIGS. 2A-2C, the reservoir section 208 the body 202 of column assembly 200 includes the reservoir 212. The reservoir section 208 may have a circular cross-sectional shape such that an exterior surface 224 of the reservoir section 208 has the shape of a cylinder, as illustrated in FIGS. 2A and 2B. However, the reservoir section 208 may have any suitable cross-sectional shape, including, for example, a polygon or an oval. The exterior surface 224 may have an outer diameter 226 when viewed perpendicular to a longitudinal axis 228 of the reservoir section 208. The reservoir section 208 may also have an interior surface 230 at least partially defining the reservoir 212, and the interior surface 230 may have an inner diameter 234 when viewed perpendicular to the longitudinal axis 228. Additionally, the reservoir section 208 may have a top portion 236 and a bottom portion 238 longitudinally opposite the top portion 236. The outer diameter 226 of the reservoir section 208 is dimensioned to be received into a standard microcentrifuge tube 38 (FIG. 3E) such that the exterior surface 224 of the reservoir section 208 mates with an interior surface 120 of the microcentrifuge tube 38, and a top surface 130 of the microcentrifuge tube 38 abuts or mates with a reservoir shoulder 240 formed at the interface between the reservoir section 208 and the collar 214, as shown in FIG. 3F. Accordingly, the outer diameter 226 of the reservoir section 208 may be between about 4 mm and 11 mm (e.g., about 8.9 mm) to accommodate a standard microcentrifuge tube 38.

As shown in FIGS. 2A and 2C, the bottom portion 238 of the reservoir section 208 may include a mating portion 242. The mating portion 242 may include a tapered surface 244 extending from the exterior surface 224 of the reservoir section 208 to a bottom coupling member 222, as illustrated in FIG. 2A. Alternatively, the mating portion 242 may include a planar surface (not shown) normal to the longitudinal axis 228 of the reservoir section 208. The mating portion 242 of the reservoir section 208 may include a support structure 245 within the reservoir 212 arranged to support the binding matrix 22. The support structure 245 may include a plurality of support ribs 246, which may be substantially identical to the plurality of support ribs 128 previously described. The plurality of support ribs 246 may be disposed within the reservoir 212 proximate to the bottom portion 238, as shown in FIG. 2C. Each of the plurality of support ribs 246 may be integrally formed with the reservoir section 208. In one embodiment, each of the plurality of support ribs 246 have a planar top surface 248 that is approximately normal to the longitudinal axis 228 of the reservoir section 208, the plurality of top surfaces 248 being configured to support the binding matrix 22 within the reservoir 212. The plurality of support ribs 246 may form a symmetrical array around a bottom passage 223 when viewed along the longitudinal axis 228 of the reservoir section 208 such that the bottom passage 223 is not obstructed. In one embodiment, the support ribs 246 will be arrayed in 45 degree intervals, as shown in FIG. 4B.

Referring to FIGS. 2A and 2C, the intermediate section 206 the body 202 of column assembly 200 includes a collar 214 integrally formed with the top portion 236 of the reservoir section 208. The collar 215 may also be secured to the top portion 236 of the reservoir section 208 by any method known in the art such as those previously mentioned. The collar 214 may have a top portion 250 and a bottom portion 252 longitudinally opposite the top portion 250. The collar 214 may have any suitable cross-sectional shape, including, for example, a polygon or an oval. The collar 214 may also have an exterior surface 254 having an outer diameter 256. The outer diameter 256 of the collar 214 is larger than the outer diameter 226 of the reservoir section 208, forming the reservoir shoulder 240 proximate to the bottom portion 252 of the collar 214 as described above. The collar 214 may have an interior surface 258 having an inner diameter 260, the interior surface 258 at least partially defining an interior portion 262 that is in flow communication with the reservoir 212. The collar 214 may also have an opening 264 proximate to the top portion 250.

As illustrated in FIGS. 2A-2C, the body 212 of column assembly 200 may also include a top coupling member 218 having a base portion 266 and a top projection 271 extending away from the base portion 266. The base portion 266 may have the general shape of a disk having an outer diameter approximately equal to the outer diameter 256 of the collar 214. The base portion 266 may have a planar bottom surface 268 configured to mate with the top portion 250 of the collar 214 and a planar top surface 270 opposite the bottom surface 268. The top projection 271 may extend from the top surface 270 of the base portion 266 along the longitudinal axis 228 of the reservoir section 208. The top projection 271 may have an interior surface 272 and an exterior surface 274 having an outer diameter 276 when viewed along the longitudinal axis 228. The top projection 271 may also have a top portion 278 and a bottom portion 280, the bottom portion 280 being proximate to the top surface 270 of the base portion 266. The top projection 271 may also include a pair of mating tabs 282 that extend radially from the top portion 278 of the top projection 271, the mating tabs 282 being functionally identical to the mating tabs 74 of the column assembly 10 previously described. An aperture 284 may be disposed on a top surface 286 of the top projection 271, and the aperture 284 and the interior surface 272 of the top projection 271 form a top passage 220. The top passage 220 may extend longitudinally through the top coupling member 218 such that, when secured to the collar 214, the top passage 220 is in flow communication with the reservoir 212 via the interior portion 216 of the collar 217. The top coupling member 218 may be secured to the collar 214 by any method known in the art, such as ultrasonic welding, adhesive bonding, interference fitting, or any combination thereof. For instance, the bottom surface 268 of the base portion 266 may be ultrasonically welded to the top portion 250 of the collar 214.

Referring again to FIGS. 2A and 2C, the lower section 210 of the body 202 of the column assembly 200 may include the bottom coupling member 222 extending away from the reservoir section 208 along the longitudinal axis 228 of the reservoir section 208. The bottom coupling member 222 may be integrally formed with the mating portion 242 of the reservoir section 208, or may be secured to the mating portion 242 by any method known in the art, such as ultrasonic welding, adhesive bonding, interference fitting, or any combination thereof. The bottom coupling member 222 may have a circular cross-sectional shape such that an exterior surface 290 of the bottom coupling member 222 has a cylindrical shape. However, the bottom coupling member 222 may have any suitable cross-sectional shape, including, for example, a polygon or an oval. The exterior surface 290 of the bottom coupling member 222 may have an outer diameter 292. An interior surface 294 of the bottom coupling member 222 may have an inner diameter 296. The outer diameter 292 of the bottom coupling member 222 may be smaller than the outer diameter 226 of the reservoir section 208. An aperture 298 formed on a bottom surface 300 of the bottom coupling member 222 and the interior surface 294 may define a bottom passage 223 extending through the bottom coupling member 222 such that the bottom passage 223 is in flow communication with the reservoir 212.

Similar to the column assembly 10 that was previously described, the column assembly 200 also allows a liquid to be drawn through the binding matrix 22 using any of several methods. First, as was the case with the column assembly 10, a syringe 28 containing a fluid can be coupled to the top projection 271 of the top coupling member 218 of column assembly 200 using a mating connection, e.g., a Luer-Lok® coupling mechanism described above and shown in FIG. 3A. The column assembly 200 may also be coupled to a reservoir adapter 29 in the same manner, as illustrated in FIG. 3C. Second, the column assembly 200 may be coupled to a vacuum manifold 34 by inserting the stopcock 132 into the bottom passage 223 of the bottom coupling member 222, as illustrated in FIG. 3B. However, an adapter (not shown), such as a tube, may also be used to couple the stopcock 132 to the bottom passage 223 of the bottom coupling member 222. Finally, the column assembly 200 can be coupled to the microcentrifuge tube 38 as described above and as shown in FIG. 3F, and the microcentrifuge tube 38 can be inserted into a centrifuge (not shown).

While various embodiments have been described above, this disclosure is not intended to be limited thereto. Variations can be made to the disclosed embodiments that are still within the scope of the appended claims.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

### Column Binding Matrices

A column binding matrix according to current disclosure (e.g., binding matrix 22 of FIG. 1C) may be composed of a solid or semi-solid (e.g., gel) matrix which allows fluid to pass through the matrix. The skilled artisan will recognize that in certain aspects a matrix is highly porous so as to maximize surface area exposed to buffer solutions and thereby the binding capacity of the matrix. A matrix can be made of various materials. Commonly used materials are dextran, cellulose, agarose and copolymers of styrene and vinylbenzene in which the divinylbenzene both cross-links the polystyrene strands and contains the charged groups. Some specific varieties of binding matrices are detailed below.

Siliceous Matrix

In certain specific embodiments, the binding matrix is a siliceous material (formed primarily of SiO₂) such glass fiber or silica beads. There are number of commercial providers of siliceous matrices, such as, for example, type GF/A, GF/B, GF/C, GF/D and GF/F matrices produced by Whatman. Such matrices are of particular use in the purification of nucleic acid molecules. Purification of DNA and RNA using these siliceous matrix materials has been previously described by Marko et al. (1987), Rikaken (1984) and Xuan et al. (1984).

Ion Exchange Matrix

Ion-exchange chromatography relies on the affinity of a substance for the matrix exchanger, which affinity depends on both the electrical properties of the material and the relative affinity of other charged substances in the buffer solution. Hence, bound material can be eluted by changing the pH, thus altering the charge of the material, or by adding competing materials, such as salts. The conditions for release vary with each bound molecular species because different substances have different electrical properties. In general, to obtain optimal separation, the methods of choice for elution are either continuous ionic strength gradient elution or stepwise elution. For an anion exchange matrix, either pH and ionic strength are gradually increased or ionic strength alone is increased. For a cation exchange matrix, both pH and ionic strength are increased. The actual choice of the elution procedure is usually a result of trial and error and of considerations of stability. For example, for unstable materials, it is best to maintain fairly constant pH.

An ion exchanger is a solid that has chemically bound charged groups to which ions are electrostatically bound; it can exchange these ions for ions in aqueous solution. Ion exchangers can be used in column chromatography to separate molecules according to charge; actually other features of the molecule are usually important so that the chromatographic behavior is sensitive to the charge density, charge distribution, and the size of the molecule.

The principle of ion-exchange chromatography is that charged molecules adsorb to ion exchangers reversibly so that molecules can be bound or eluted by changing the ionic environment. Separation on ion exchangers is usually accomplished in two stages: first, the substances to be separated are bound to the matrix, using conditions that give stable and tight binding; then the column is eluted with buffers of different pH, ionic strength, or composition and the components of the buffer compete with the bound material for the binding sites.

An ion matrix or exchanger is usually a three-dimensional network that contains covalently linked charged groups. If a group is negatively charged, it will exchange positive ions and is a cation exchanger. A typical group used in cation exchangers is the sulfonic group, SO₃⁻. If an H⁺ is bound to the group, the exchanger is said to be in the acid form; it can, for example, exchange on H⁺ for one Na⁺ or two H⁺ for one Ca²⁺. The sulfonic acid group is a strongly acidic cation exchanger. Other commonly used groups are phenolic hydroxyl and carboxyl, both weakly acidic cation exchangers. If the charged group is positive—for example, a quaternary amino group—it is a strongly basic anion exchanger. The most common weakly basic anion exchangers are aromatic or aliphatic amino groups. Table 1 gives the composition of many ion exchangers.

The total capacity of an ion exchanger measures its ability to take up exchangeable groups per milligram of dry weight. This number is supplied by the manufacturer and is important because, if the capacity is exceeded, ions will pass through the column without binding.

TABLE 1

| Matrix | Exchanger | Functional Group | Tradename |
|---|---|---|---|
| Dextran | Strong Cationic | Sulfopropyl | SP-Sephadex |
| | Weak Cationic | Carboxymethyl | CM-Sephadex |
| | Strong Anionic | Diethyl-(2- | QAE-Sephadex |
| | | hydroxypropyl)- | |
| | | aminoethyl | |
| | Weak Anionic | Diethylaminoethyl | DEAE-Sephadex |
| Cellulose | Cationic | Carboxymethyl | CM-Cellulose |
| | Cationic | Phospho | P-cel |
| | Anionic | Diethylaminoethyl | DEAE-cellulose |
| | Anionic | Polyethylenimine | PEI-Cellulose |
| | Anionic | Benzoylated- | DEAE(BND)- |
| | | naphthoylated, | cellulose |
| | | deiethylaminoethyl | |
| | Anionic | p-Aminobenzyl | PAB-cellulose |
| Styrene-divinyl- | Strong Cationic | Sulfonic acid | AG 50 |
| benzene | Strong Anionic | | AG 1-Source15Q |
| | Strong Cationic + | Sulfonic acid + | AG 501 |
| | Strong Anionic | Tetramethylammonium | |
| Acrylic | Weak Cationic | Carboxylic | Bio-Rex 70 |
| | Strong Anionic | Trimethylamino- | E. Merk |
| | | ethyl | |
| | Strong Anionic | Trimethylamino | Toso Haas TSK-Gel- |
| | | group | Q-5PW |
| Phenolic | Strong Cationic | Sulfonic acid | Bio-Rex 40 |
| Expoxyamine | Weak Anionic | Tertiary amino | AG-3 |

The available capacity is the capacity under particular experimental conditions (i.e., pH, ionic strength). For example, the extent to which an ion exchanger is charged depends on the pH (the effect of pH is smaller with strong ion exchangers). Another factor is ionic strength because small ions near the charged groups compete with the sample molecule for these groups. This competition is quite effective if the sample is a macromolecule because the higher diffusion coefficient of the small ion means a greater number of encounters. Clearly, as buffer concentration increases, competition becomes keener.

The porosity of the matrix is an important feature because the charged groups are both inside and outside the matrix and because the matrix also acts as a molecular sieve. Large molecules may be unable to penetrate the pores; so the capacity will decease with increasing molecular dimensions. The porosity of the polystyrene-based resins is determined by the amount of cross-linking by the divinylbenzene (porosity decreases with increasing amounts of divinylbenzene). With the Dowex and AG series, the percentage of divinylbenzene is indicated by a number after an X—hence, Dowex 50-X8 is 8% divinylbenzene

Ion exchangers come in a variety of particle sizes, called mesh size. Finer mesh ion exchange resins have an increased surface-to-volume ratio and therefore increased capacity and decreased time for exchange to occur for a given volume of the exchanger. On the other hand, fine mesh produces a slow flow rate, which can increase diffusional spreading.

There are a number of choices to be made when employing ion exchange chromatography as a technique. The first choice to be made is whether the exchanger is to be anionic or cationic. If the materials to be bound to the column have a single charge (i.e., either plus or minus), the choice is clear. However, many substances (e.g., proteins, viruses), carry both negative and positive charges and the net charge depends on the pH. In such cases, the primary factor is the stability of the substance at various pH values. Most proteins have a pH range of stability (i.e., in which they do not denature) in which they are either positively or negatively charged. Hence, if a protein is stable at pH values above the isoelectric point, an anion exchanger should be used; if stable at values below the isoelectric point, a cation exchanger is required.

The choice between strong and weak exchangers is also based on the effect of pH on charge and stability. For example, if a weakly ionized substance that requires very low or high pH for ionization is chromatographed, a strong ion exchanger is called for because it functions over the entire pH range. However, if the substance is labile, weak ion exchangers are preferable because strong exchangers are often capable of distorting a molecule so much that the molecule denatures. The pH at which the substance is stable must, of course, be matched to the narrow range of pH in which a particular weak exchanger is charged. Weak ion exchangers are also excellent for the separation of molecules with a high charge from those with a small charge, because the weakly charged ions usually fail to bind. Weak exchangers also show greater resolution of substances if charge differences are very small. If a macromolecule has a very strong charge, it may be impossible to elute from a strong exchanger and a weak exchanger again may be preferable. In general, weak exchangers are more useful than strong exchangers.

The Sephadex and Bio-gel exchangers offer a particular advantage for macromolecules that are unstable in low ionic strength. Because the cross-linking in the support matrix of these materials maintains the insolubility of the matrix even if the matrix is highly polar, the density of ionizable groups can be made several times greater than is possible with cellulose ion exchangers. The increased charge density introduces an increased affinity so that adsorption can be carried out at higher ionic strengths. On the other hand, these exchangers retain some of their molecular sieving properties so that sometimes molecular weight differences annul the distribution caused by the charge differences; the molecular sieving effect may also enhance the separation.

Small molecules are best separated on matrices with small pore size (i.e., the underlying support matrix has a high degree of cross-linking) because the available capacity is large, whereas macromolecules need large pore size. However, except for the Sephadex type matrices, most ion exchange media do not afford the opportunity for matching the porosity with the molecular weight.

The cellulose ion exchangers have proved to be the most effective for purifying large molecules such as proteins and polynucleotides. This is because the matrix is fibrous, and hence all functional groups are on the surface and available to even the largest molecules. In many cases, however, beaded forms such as DEAE-Sephacel and DEAE-Biogel P are more useful because there is a better flow rate and the molecular sieving effect aids in separation.

Selecting a mesh size has attendant difficulties. Small mesh size improves resolution but decreases flow rate, which increases zone spreading and decreases resolution. Hence, the appropriate mesh size is usually determined empirically.

Buffers themselves consist of ions, and therefore, they can also exchange, and the pH equilibrium can be affected. To avoid these problems, the rule of buffers is adopted: use cationic buffers with anion exchangers and anionic buffers with cation exchangers. Because ionic strength is a factor in binding, a buffer should be chosen that has a high buffering capacity so that its ionic strength need not be too high. Furthermore, for best resolution, it has been generally found that the ionic conditions used to apply the sample to the column (starting conditions) should be near those used for eluting the column.

Affinity Matrices

Affinity chromatography employing an affinity matrix is used to separate molecules or complexes by selective adsorption onto and/or elution from a solid medium, generally in the form of a column. The solid medium is usually an inert carrier matrix to which is attached a ligand having the capacity to bind, under certain conditions, the required protein or proteins in preference to others present in the same sample, although in some cases the matrix itself may have such selective binding capacity. The ligand may be biologically complementary to the protein to be separated, for example, antigen and antibody, or may be any biologically unrelated molecule which by virtue of the nature and steric relationship of its active groups has the power to bind the protein. Examples of commonly used affinity chromatography include immobilized metal affinity chromatography (IMAC), sulfated affinity chromatography, dye affinity chromatography, and heparin affinity. In another example, the chromatographic medium may be prepared using one member of a binding pair, e.g., a receptor/ligand binding pair, or antibody/antigen binding pair (immunoaffinity chromatography).

The support matrices commonly used in association with protein-binding ligands employed in affinity chromatography include, for example, polymers and copolymers of agarose, dextrans and amides, especially acrylamide, or glass beads or nylon matrices. Cellulose and substituted celluloses are generally found unsuitable when using dyes, since, although they bind large amounts of dye, the dye is poorly accessible to the protein, resulting in poor protein binding. Other support matrices also may be used. Exemplary affinity chromatographic techniques are discussed in further detail below.

Immobilized metal affinity chromatography (IMAC), also known as metal chelate affinity chromatography (MCAC), is used primarily in the purification of polyhistidine tagged recombinant proteins. This is achieved by using the natural tendency of histidine to chelate divalent metals. Placing the metal ion on a chromatographic support allows purification of the histidine tagged proteins. This is a highly efficient method that has been employed by those of skill in the art for a variety of protein purification methods.

The high efficiency of the IMAC method is based on the interaction of a covalently bound chelating ligand immobilized on a chromatographic support with histidine-containing proteins. In this method, the metal ion must have a high affinity for the support. Commonly used as the supporting matrix are iminodiacetic acid derivatives.

Those of skill in the art are referred to U.S. Pat. No. 4,431,546 which describes in detail methods of metal affinity chromatographic separation of biological or related substances from a mixture. The chromatographic media described in the aforementioned patent comprise binding materials which have a ligand containing at least one of the groups anthraquinone, phthalocyanine or aromatic azo, in the presence of at least one metal ion selected from the group Ca²⁺, Sr²⁺, Ba²⁺, Al³⁺, Co²⁺, Ni²⁺, Cu²⁺ or Zn²⁺. In IMAC techniques used herein, the ligand may be linked directly to the matrix or via a spacer arm. The process may be performed at atmospheric pressure or under pressure, especially high pressure (100-3500 psi).

As with all the chromatographic techniques, the nature of the contact, washing and eluting solutions for IMAC depends on the substance to be separated. Generally the contact solution is made up of the substance to be separated and a metal salt dissolved in a buffer solution, while the washing solution comprises the same metal salt dissolved in the same buffer. The eluting solution, may be a buffer solution, either alone or containing a chelating agent or it may be an alkali metal salt or a specific desorbing agent. Alternatively the eluting solution may be a mixture of two or more of these solutions or two or more of these solutions used consecutively.

The most common chelating group used in this technique is iminodiacetic acid (IDA). It is coupled to a matrix such as Sepharose™ 6B, via a long hydrophilic spacer arm. The spacer arm ensures that the chelating metal is fully accessible to all available binding sites on a protein. Affiland (Ans-Liege, Belgium) is one exemplary commercial source of immobilized iminodiacetic acid (IDA), nitrilotriacetic acid (NTA) and a pentadentate chelator (PDC) ligand for IMAC. Briefly, immobilized IDA is a tridentate ligand at physiological pH, NTA is a pentadentate ligand at basic pH and a tridentate ligand at pH 8.0. In the presence of the electron donor cross-linkers, immobilized IDA forms octahedral complexes with polyvalent metal ions including Cu²⁺, Zn²⁺, Ni²⁺ and Co²⁺. This column has a selective binding for histidine-containing proteins. The elution of histidine-containing proteins uses a high concentration of Imidazole.

The IDA matrix is supplied bound to a number of underlying matrices e.g., Sepharose, and the like. The ISA-matrix is degassed and then applied to a column and washed with 10 volumes of distilled water. The bivalent or trivalent cation is then applied to the washed matrix in a distilled water at a concentration 5 mg/ml in distilled water, at a flow rate of 50 ml/cm²/hour, until saturation. The metal chelate affinity matrix is then equilibrated with an appropriate buffer e.g., Tris 50 mM, AcOH pH 8.0. The equilibrated column is then ready for use.

Fractogel® EMD chelate iminodiacetic acid is an IMAC matrix supplied by VWR International, Merck (Poole, Dorset, U.K.). TALON™ resin is a durable IMAC resin that uses cobalt ions for purifying recombinant polyhistidine-tagged proteins (Clontech, Palo Alto, Calif.).

Another common chelating group for IMAC applications is tris(carboxymethyl)-ethylenediamine (TED). TED gels show stronger retention of metal ions and weaker retention of proteins relative as compared to IDA-based matrices. TED matrices form a complex (single coordination site) whereas IDA matrices form a chelate (multiple coordination sites). The most commonly used metals for IMAC are zinc and copper; however, nickel cobalt, and calcium have also been used successfully.

Suitable immobilized metal affinity media include, Chelating Sepharose Fast Flow (Amersham Biosciences AB, Uppsala Sweden), HiTrap Chelating Media (Sigma-Aldrich, St. Louis, Mo.), and TSKgel Chelate-5PW (Sigma-Aldrich, St. Louis, Mo.).

Sulfated affinity chromatography uses oligosaccharide (generally cellulose) resins as support matrices. These resins are derivatized with a sulfate compound. The sulfated affinity chromatographic medium attracts certain surface proteins or contaminants that are attracted to sulfate. Prussak, U.S. Pat. No. 5,447,859, describes the use of sulfated affinity media in the purification of viruses. Suitable sulfated affinity media include, Matrex Cellufine Sulfate Affinity Media (Millipore, Bedford, Mass.), and Sterogene Sulfated Hi Flow (Carlsbad, Calif.)

Dye affinity chromatography employs a matrix which comprises a dye bound to the underlying column matrix. Proteins have been successfully isolated using this chromatographic technique which relies on an interaction between the protein and the dye molecule. The mechanism by which such interactions occur are not well known but it is thought that some dyes mimic cofactors and/or substrates of the proteins being retained by the column.

A variety of dye affinity media are available for dye-affinity chromatography, including but not limited to MIMETIC Red™ 2 A6XL, MIMETIC Red™ 3 A6XL, MIMETIC Blue™ 1 A6XL, MIMETIC Blue™ 2 A6XL, MIMETIC Orange™ 1 A6XL, MIMETIC Orange™ 2 A6XL, MIMETIC Orange™ 3 A6XL, MIMETIC Yellow™ 1 A6XL, MIMETIC Yellow™ 2 A6XL, and MIMETIC Green™ 1 A6XL (Affinity Chromatography Ltd., Freeport, Great Britain). These media are 6% cross-linked agarose beads, 45-164 μn, to which a dye ligand is linked via a spacer arm. Those of skill in the art will understand that the above-discussed dye ligands are only an exemplary list and other dye ligands are widely available for dye-affinity chromatography. For example, other available dye-affinity chromatography media include but are not limited to Fast Flow Blue Sepharose 6 (Amersham Biosciences AB, Uppsala Sweden), Fast Flow Q-Sepharose (Amersham Biosciences AB, Uppsala Sweden), Blue Trisacryl (Ciphergen Biosystems, Fremont, Calif.), and Blue Sepharose FF (Amersham Biosciences AB, Uppsala Sweden). Selective triazinyl protein-binding dyes such as Procion Scarlet™ MX-G; Procion Yellow™ H-A; Procion Turquoise™ MX-G; Procion Red™ MX-5B; Procion Blue™ MX-R; Procion Red™ MX-2B; Procion Yellow™ MX-6G also may be used in a dye affinity chromatographic method of the present invention.

Proteins bind to dye ligands under physiological conditions (slightly alkaline pH and salt concentration of approximately 150 mM), obviating the need to adjust pH and ionic strength of the CCL prior to application to these chromatographic media. The bound proteins can be eluted using increased salt concentration, increased pH, denaturing agents, or combinations thereof.

Any of the chromatography steps (dye affinity or other chromatography) discussed herein may be carried out this step in the cold (e.g., 4°-10° C.) to minimize the likelihood of bacterial contamination, however, for large scale production of viral preparations as described herein the steps also may be conducted at room temperature. Methods for determining the binding specificity of dye-ligand affinity media and elution conditions suitable for protein binding are known in the art and include the use of commercially available assay kits (e.g., PIKSI™ test kit available from Affinity Chromatography Ltd.). See, for example, Kroviarski et al., J. Chromatography 449:403-412 (1988) and Miribel et al., J. Biochem. Biophys. Methods, 16:1-16 (1988). Of course, those of skill in the art will be able to make dye affinity media simply by adhering a selected dye to a given matrix such as agarose, dextrans, cellulose and amides, glass beads, nylon matrices, styrene-divinyl-benzene, and the like.

U.S. Pat. No. 4,016,149 and Baird et al., FEBS Letter, Vol. 70 (1976) page 61, describe solid media wherein the ligands are mono-chloro-triazinyl dyes and are bound to dextran or agarose matrices by substitution at the chloride group. While binding in alkaline buffered media results in low protein binding capacity, it is possible to increase the dye binding by cyanogen bromide activation of the agarose matrix. However, cyanogen bromide activation has serious disadvantages, especially for industrial and biological use.

U.K. Patent No. 2,015,552 describes a method of achieving useful controlled levels of dye binding without the use of cyanogen bromide, by a process comprising reacting a protein-binding ligand material containing chlorotriazinyl or related groups with an aqueous suspension of a non-cellulosic matrix containing free hydroxy or amino groups in the presence of an alkali metal hydroxide at least pH 8, and subsequently washing the resulting solid medium to remove unreacted dye.

Protein-binding ligands described in U.K. Pat. No. 2,015,552 include material containing a mono or dichloro triazinyl group or related group, in particular, the so-called triazinyl dyes such as those sold under the trade marks “Cibacron” and “Procion”. These are normally triazinyl derivatives of sulphonated anthraquinones, phthalocyanines or polyaromatic azo compounds discussed in U.S. Pat. No. 4,623,625, incorporated herein by reference.

U.S. Pat. No. 4,623,625 discusses that different triazinyl dyes bound to an agarose matrix are specific for different proteins in a given extract. It may be useful in the present invention to apply the CCL to a dye affinity chromatography medium made with a selected dye to remove a specific set of contaminating proteins. Alternatively, the CCL may be applied to a succession of dye affinity chromatographic media each of a different selected dye, in a suitable buffer at a pH between pH 5.6-6.0 and containing about 5 to 20 mg/ml protein.

Immunoaffinity column chromatography involves the preparation of a column media in which the matrix of the chromatographic medium is linked to an antibody or an antigen, that can specifically bind the target species (i.e., antigen or antibody, respectively) from a complex mixture. Immunoaffinity chromatography is specific for the species of interest being isolated and may be performed under mild conditions. Immunoaffinity purification techniques are well known in the art (see, Harlow, et al., Antibodies: A Laboratory Manual, Cold Spring Harbor Laboratory Press: 511-552 (1988)).

Heparin affinity media is another commonly used affinity chromatography. Heparin has two properties that facilitate its use in chromatographic techniques. It can act as an affinity ligand, for example, in its interaction with coagulation factors, or heparin can function as a high capacity cation exchanger, due to its anionic sulfate groups. Gradient elution with salt is most commonly used in both cases to elute the bound species from the column. Suitable heparin affinity media include but are not limited to Heparin Sepharose 6 Fast Flow (Amersham Biosciences AB, Uppsala Sweden), HiTrap Heparin HP (Amersham Biosciences AB, Uppsala Sweden), and Cellufine Heparin (Millipore, Bedford, Mass.).

Other chromatographic media commonly used in affinity chromatography include e.g., hydroxyapetite media (e.g., BioRad MacroPrep Ceramic Hydroxyapatite). Such media may also be useful in the methods of the present invention.

Size Exclusion Matrices

Size exclusion chromatography, otherwise known as gel filtration or gel permeation chromatography, relies on the penetration of macromolecules in a mobile phase into the pores of stationary phase particles. Differential penetration of the macromolecules is a function of the hydrodynamic volume of the particles. Size exclusion media exclude larger molecules from the interior of the particles while the smaller molecules are accessible to this volume. The order of elution can be predicted by the size of the protein as a linear relationship exists between elution volume and the log of the molecular weight of the protein being eluted.

Hydrophobic Interaction Matrices

Certain proteins are retained on affinity columns containing hydrophobic spacer arms.

This observation is exploited in the technique of hydrophobic interaction chromatography (HIC). Hydrophobic adsorbents now available include octyl or phenyl groups. Hydrophobic interactions are strong at high solution ionic strength, as such the CCL samples need not be desalted before application to the adsorbent. Elution is achieved by changing the pH or ionic strength or by modifying the dielectric constant of the eluant using, for instance, ethanediol. A recent introduction is cellulose derivatized to introduce even more hydroxyl groups. This material (Whatman HB1, Whatman Inc., New Jersey, USA) is designed to interact with proteins by hydrogen bonding. Samples are applied to the matrix in a concentrated (over 50% saturated, >2M) solution of ammonium sulphate. Proteins are eluted by diluting the ammonium sulphate. This introduces more water which competes with protein for the hydrogen bonding sites.

A further detailed description of the general principles of hydrophobic interaction chromatography media may be found in U.S. Pat. No. 3,917,527 and in U.S. Pat. No. 4,000,098. The application of HIC to the purification of specific proteins is exemplified by reference to the following disclosures: human growth hormone (U.S. Pat. No. 4,332,717), toxin conjugates (U.S. Pat. No. 4,771,128), antihemolytic factor (U.S. Pat. No. 4,743,680), tumor necrosis factor (U.S. Pat. No. 4,894,439), interleukin-2 (U.S. Pat. No. 4,908,434), human lymphotoxin (U.S. Pat. No. 4,920,196) and lysozyme species (Fausnaugh, J. L. and F. E. Regnier, J. Chromatog. 359:131-146 (1986)) and soluble complement receptors (U.S. Pat. No. 5,252,216). Suitable hydrophobic interaction chromatography media include, Pharmacia's phenyl-Sepharose, and Tosohaas' butyl, phenyl and ether Toyopearl 650 series resins.

In certain aspects, methods and kits for chromatographic purification employing a column described herein are provided. Embodiments described below concern elements for use in such methods and/or for inclusion in such a purification kit.

### Buffer and Solution Formulation

In certain embodiments, chromatographic purification methods described herein employ solutions such as sample solutions or suspensions, binding buffers, washing buffers and/or elution buffers. As used herein a “binding buffer” refers to a buffer formulated to allow a compound (or, in some cases, one or more impurities) to bind to a binding matrix in a column in a given temperature range. In certain aspects, a sample may be comprised in a solution or suspension which acts as a binding buffer or a solution may be added to the sample to facilitate binding to a column matrix. A “wash buffer” refers to a buffer formulated to allow a compound (or a substantial amount of the compound) to remain bound to a binding matrix in a column in a given temperature range. Moreover a wash buffer may be formulated to allow elution of one or more contaminants from the column binding matrix, while not substantially eluting the bound compound. An “elution buffer” means to a buffer formulated to cause release of a compound from a binding matrix in a given temperature range so that the compound can be eluted through the column. The skilled artisan will recognize buffer may be formulated in various ways that affect the ability of compounds and/or impurities and contaminants to bind to a column binding matrix. For example, buffers may be an aqueous buffer formulated with different concentrations of salts, detergents, chaotropic agents, viscosity altering agents or other additives. Moreover, the skilled worker will recognize that other factors such as temperature and turbulence in fluid flow will affect the binding characteristic of a column binding matrix and buffer formulations may be adjusted to compensate for these factors. In certain aspects other buffers such as preparative buffers are described herein. Preparative buffers may be used to solubilize or concentrate components for purification from, for example, cells, solid objects, body fluids and complex mixtures such as soils.

pH Buffering Agents

In certain aspects buffer solutions for use according to the disclosure may comprise one or more agents to regulate or buffer pH of the solution. For example, some common pairs of buffering agents for use in chemical applications include but are not limited to HCl/sodium citrate, citric acid/sodium citrate, acetic acid/sodium acetate, Na₂HPO₄/NaH₂PO₄, and Borax/sodium hydroxide. Buffering agents that are more commonly used in biological applications include TAPS (3-{[tris(hydroxymethyl)methyl]amino}propanesulfonic acid), Bicine (N,N-bis(2-hydroxyethyl)glycine), Tris(tris(hydroxymethyl)methylamine), Tricine (N-tris(hydroxymethyl)methylglycine), HEPES (4-2-hydroxyethyl-1-piperazineethanesulfonic acid), TES 7(2-{[tris(hydroxymethyl)methyl]amino}ethanesulfonic acid), MOPS (3-(N-morpholino)propanesulfonic acid), PIPES (piperazine-N,N′-bis(2-ethanesulfonic acid)), Cacodylate (dimethylarsinic acid) and MES (2-(N-morpholino)ethanesulfonic acid). The choice of a buffering agent (or combination of buffer agents) for any particular application will depend on the desired pH range for the solution and can be readily determined by a person of skill in the art.

Salts

Salts are agents that may be used in aqueous buffer solutions to alter the ionic strength of the solution. In certain aspects, specific salts may be added to solution to alter the concentration of particular anions and cations in the solution and alter the binding properties of the matrix in contact withy the solution. Some common salt-forming cations include, but are not limited to, ammonium (NH₄⁺), Calcium (Ca₂⁺) Iron (Fe₂⁺ and Fe₃⁺), Magnesium (Mg₂⁺) and Sodium (Na⁺). Common salt-forming anions include, but are not limited to, Acetate CH₃COO⁻, Carbonate CO₃²⁻, Chloride CV, Citrate HOC(COO⁻)(CH₂COO⁻)₂, Hydroxide OH⁻, Nitrate NO₃⁻, Nitrite NO₂⁻, Phosphate PO₄³⁻ and Sulfate SO₄²⁻.

Detergents

Detergents are amphipathic molecules with an apolar end of aliphatic or aromatic nature and a polar end which may be charged or uncharged. Detergents are more hydrophilic than lipids and thus have greater water solubility than lipids. They allow for the dispersion of water insoluble compounds into aqueous media and are used to isolate and purify proteins in a native form. In certain aspects, detergents may be used in preparative buffer, for example for lysing cell membranes. One of skill in the art would be familiar with the wide range of detergents available for lysing cells. Detergents can be denaturing or non-denaturing. The former can be anionic such as sodium dodecyl sulfate or cationic such as ethyl trimethyl ammonium bromide. These detergents totally disrupt membranes and denature the protein by breaking protein-protein interactions. Non denaturing detergents can be divided into non-anionic detergents such as Triton® X-100, bile salts such as cholates and zwitterionic detergents such as CHAPS. Zwitterionics contain both cationic and anion groups in the same molecule, the positive electric charge is neutralized by the negative charge on the same or adjacent molecule. Moreover, detergents may be used in binding, washing or elution buffers.

Denaturing agents such as SDS bind to proteins as monomers and the reaction is equilibrium driven until saturated. Thus, the free concentration of monomers determines the necessary detergent concentration. SDS binding is cooperative i.e. the binding of one molecule of SDS increase the probability of another molecule binding to that protein, and alters proteins into rods whose length is proportional to their molecular weight.

Non-denaturing agents such as Triton® X-100 do not bind to native conformations nor do they have a cooperative binding mechanism. These detergents have rigid and bulky apolar moieties that do not penetrate into water soluble proteins. They bind to the hydrophobic parts of proteins. Triton® X100 and other polyoxyethylene nonanionic detergents are inefficient in breaking protein-protein interaction and can cause artifactual aggregations of protein. These detergents will, however, disrupt protein-lipid interactions but are much gentler and capable of maintaining the native form and functional capabilities of the proteins.

In certain aspects detergents used in preparative o buffers are removed prior to chromatiographic purification. Dialysis, for instance, can be employed with detergents that exist as monomers. Dialysis is somewhat ineffective with detergents that readily aggregate to form micelles because they micelles are too large to pass through dialysis. Ion exchange chromatography can be utilized to circumvent this problem. The disrupted protein solution is applied to an ion exchange chromatography column and the column is then washed with buffer minus detergent. The detergent will be removed as a result of the equilibration of the buffer with the detergent solution. Alternatively the protein solution may be passed through a density gradient. As the protein sediments through the gradients the detergent will come off due to the chemical potential.

Often a single detergent is not versatile enough for the solubilization and analysis of the milieu of proteins found in a cell. The proteins can be solubilized in one detergent and then placed in another suitable detergent for protein analysis. The protein detergent micelles formed in the first step should separate from pure detergent micelles. When these are added to an excess of the detergent for analysis, the protein is found in micelles with both detergents. Separation of the detergent-protein micelles can be accomplished with ion exchange or gel filtration chromatography, dialysis or buoyant density type separations.

Triton® X-Detergents

This family of detergents (Triton® X-100, X114 and NP-40) have the same basic characteristics but are different in their specific hydrophobic-hydrophilic nature. All of these heterogeneous detergents have a branched 8-carbon chain attached to an aromatic ring. This portion of the molecule contributes most of the hydrophobic nature of the detergent. Triton®X detergents are used to solubilize membrane proteins under non-denaturing conditions. The choice of detergent to solubilize proteins will depend on the hydrophobic nature of the protein to be solubilized. Hydrophobic proteins require hydrophobic detergents to effectively solubilize them.

Triton®X-100 and NP-40 are very similar in structure and hydrophobicity and are interchangeable in most applications including cell lysis, delipidation protein dissociation and membrane protein and lipid solubilization. Generally 2 mg of detergent is used to solubilize 1 mg membrane protein or 10 mg detergent/1 mg of lipid membrane. Triton® X-114 is useful for separating hydrophobic from hydrophilic proteins.

Brij® Detergents

These are similar in structure to Triton®X detergents in that they have varying lengths of polyoxyethylene chains attached to a hydrophobic chain. However, unlike Triton®X detergents, the Brij® detergents do not have an aromatic ring and the length of the carbon chains can vary. The Brij® detergents are difficult to remove from solution using dialysis but may be removed by detergent removing gels. Brij® 58 is most similar to Triton® X100 in its hydrophobic/hydrophilic characteristics. Brij®-35 is a commonly used detergent in HPLC applications.

Dializable Nonionic Detergents

η-Octyl-β-D-glucoside (octylglucopyranoside) and η-Octyl-β-D-thioglucoside (octylthioglucopyranoside, OTG) are nondenaturing nonionic detergents which are easily dialyzed from solution. These detergents are useful for solubilizing membrane proteins and have low UV absorbances at 280 nm. Octylglucoside has a high CMC of 23-25 mM and has been used at concentrations of 1.1-1.2% to solubilize membrane proteins.

Octylthioglucoside was first synthesized to offer an alternative to octylglucoside. Octylglucoside is expensive to manufacture and there are some inherent problems in biological systems because it can be hydrolyzed by β-glucosidase.

Tween® Detergents

The Tween® detergents are nondenaturing, nonionic detergents. They are polyoxyethylene sorbitan esters of fatty acids. Tween® 20 and Tween® 80 detergents are used as blocking agents in biochemical applications and are usually added to protein solutions to prevent nonspecific binding to hydrophobic materials such as plastics or nitrocellulose. Generally, these detergents are used at concentrations of 0.01-1.0% to prevent nonspecific binding to hydrophobic materials.

The difference between these detergents is the length of the fatty acid chain. Tween® 80 is derived from oleic acid with a C18 chain while Tween® 20 is derived from lauric acid with a C12 chain. The longer fatty acid chain makes the Tween® 80 detergent less hydrophilic than Tween® 20 detergent. Both detergents are very soluble in water.

The Tween® detergents are difficult to remove from solution by dialysis, but Tween® 20 can be removed by detergent removing gels. The polyoxyethylene chain found in these detergents makes them subject to oxidation (peroxide formation) as is true with the Triton® X and Brij® series detergents.

Zwitterionic Detergents

The zwitterionic detergent, CHAPS, is a sulfobetaine derivative of cholic acid. This zwitterionic detergent is useful for membrane protein solubilization when protein activity is important. This detergent is useful over a wide range of pH (pH 2-12) and is easily removed from solution by dialysis due to high CMCs (8-10 mM). This detergent has low absorbances at 280 nm making it useful when protein monitoring at this wavelength is necessary. CHAPS is compatible with the BCA Protein Assay and can be removed from solution by detergent removing gel. Proteins can be iodinated in the presence of CHAPS and CHAPS has been successfully used to solubilize intrinsic membrane proteins and receptors and maintain the functional capability of the protein.

Chaotroptic Agents

In certain aspects buffer formulations may comprise chaotropic agents such as urea, guanidinium chloride or lithium perchlorate. For example, methods for purification of nucleic acid molecules may employ a binding buffer formulated with a chaotropic agent that faculties nucleic acid binding to a silica matrix. In certain aspects the chaotropic agent is formulated to at concentration sufficient to denture most biological molecules (e.g., 6M or greater Urea, 6M or greater guanidinium chloride or about 4.5M or greater lithium perchlorate).

### EXAMPLES

The following examples are included to demonstrate certain embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Isolation of Plasmid DNA Using a Vacuum Manifold

The following procedure is performed at room temperature. Ensure that the 7× lysis Buffer has not precipitated during shipping. To completely resuspend the buffer, incubate the bottle at 30-37° C. for 30 minutes and mix by inversion.

1. Add 6 ml of bacterial culture to a 50 ml conical tube. (Alternatively, centrifuge up to 35 ml of bacterial culture in a 50 ml conical tube for 10 minutes at 3,400×g. Discard the supernatant. Add 6 ml of TE or water to the bacterial cell pellet and completely resuspend by vortexing or pipetting.)

2. Add 1 ml of 7× lysis Buffer to 1-5 samples and mix by inverting the tube 4-6 times. Proceed to step 3 within 2 minutes. (Excessive lysis can result in denatured plasmid DNA. If processing a large number of samples, we recommend working with groups of five or less at a time. Continue with the next set of five samples after the first set has been neutralized and mixed thoroughly.)

3. Add 3.5 ml of cold Neutralization Buffer (with RNase A) and mix thoroughly. Invert the sample an additional 2-3 times to ensure complete neutralization.

4. Lock the reservoir adapter (78 in FIG. 3C) with filter to the top of the universal column and attach the assembly onto a vacuum manifold (FIG. 3B illustrates the universal column attached to a vacuum manifold).

5. Add the entire buffer mixture into reservoir adapter (e.g., the blue Zymo-Midi Filter™ column), let the cell debris float to the surface and turn on the vacuum until all of the liquid has passed completely through the column assembly.

6. Remove and discard the reservoir adapter from the top of the universal column.

7. Add 600 μl of Endo-Wash Buffer to the universal column and turn on the vacuum until all of the liquid passes completely through the column.

8. Add 600 μl of Zyppy™ Wash Buffer and turn on the vacuum until all of the liquid passes completely through the column. Repeat this step.

9. Vacuum for an additional 2 minutes to remove all residual Zyppy™ Wash Buffer. Alternatively, the three washes (steps 7-9) can be performed by either:
(i) placing the universal column (e.g., column 10 of FIGS. 1A-1C) into a collection tube and centrifuging at 11,000×g for one minute (see, e.g., FIG. 3E-F) (Empty the collection tube after each wash to prevent contamination of the spin column or
(ii) pushing the wash buffer through the universal column by attaching the column to a syringe and applying pressure (see, e.g., FIG. 3A).

10. Transfer the universal column into a clean 1.5 nil microcentrifuge tube then add 150 μl of Zyppy Elution Buffer to the center of the column (Elution Buffer contains 0.1 mM EDTA; if required, pure water can also be used to elute the DNA). Incubate at room temperature for one minute, then centrifuge at 11,000×g for 30 seconds to elute the plasmid DNA. Alternatively, the DNA may be eluted by applying the elution buffer to the column, at room temperature for one minute and pushing the wash buffer through the universal column by attaching the column to a syringe and applying pressure. The eluted DNA may be captured in a microcenterfuge tube or other convenient storage container.

### Example 2

### Isolation of Plasmid DNA Using a Centrifuge

1. Add 6 ml of bacterial culture to a 50 ml conical tube. (Alternatively, centrifuge up to 35 ml of bacterial culture in a 50 ml conical tube for 10 minutes at 3,400×g. Discard the supernatant. Add 6 ml of TE or water to the bacterial cell pellet and completely resuspend by vortexing or pipetting.)

2. Add 1 ml of 7× Lysis Buffer to 1-5 samples and mix by inverting the

tube 4-6 times. Proceed to step 3 within 2 minutes to avoid excessive lysis and plasmid denaturation.

3. Add 3.5 ml of cold Neutralization Buffer (with RNase A) and mix thoroughly. The sample will turn cloudy when the neutralization is complete and a precipitate will form. Invert the sample an additional 2-3 times to ensure complete neutralization.

4. Lock the reservoir adapter (78 in FIG. 3C) with filter to the top of the universal column and position the assembly into a clean 50 ml conical tube (see, e.g., FIG. 3D).

5. Add the entire buffer mixture into reservoir adapter (e.g., the blue Zymo-Midi Filter™ column), place the cap on the conical tube, and centrifuge at 500×g for 6 minutes.

6. Remove and discard the reservoir adapter from the top of the universal column.

7. Transfer the universal column to a collection tube (see, e.g., FIG. 3E-F).

8. Add 600 μl of Endo-Wash Buffer to the universal column and centrifuge in a microcentrifuge at 11,000×g for 30 seconds. Discard the flowthrough.

9. Add 600 μl of Zyppy™ Wash Buffer a centrifuge in a microcentrifuge at 11,000×g for 1 minute. Discard the flowthrough and repeat this step. Alternatively, the three washes (steps 8-9) can be performed by either:
(i) attaching the to the universal column to a vacuum manifold and allowing the buffer from each wash to passes completely through the column (vacuum for an additional 2 minutes to remove all residual buffer) or
(ii) pushing the wash buffer through the universal column by attaching the column to a syringe and applying pressure (see, e.g., FIG. 3A).

10. Transfer the universal column into a clean 1.5 ml microcentrifuge tube then add 150 μl of Zyppy Elution Buffer to the center of the column (Elution Buffer contains 0.1 mM EDTA; if required, pure water can also be used to elute the DNA). Incubate at room temperature for one minute, then centrifuge at 11,000×g for 30 seconds to elute the plasmid DNA. Alternatively, the DNA may be eluted by applying the elution buffer to the column, at room temperature for one minute and pushing the wash buffer through the universal column by attaching the column to a syringe and applying pressure. The eluted DNA may be captured in a microcenterfuge tube or other convenient storage container.

### REFERENCES

Each of the foregoing documents is hereby incorporated by reference in its entirety:
U.K. Patent No. 2,015,552
U.S. Pat. No. 3,917,527
U.S. Pat. No. 4,000,098
U.S. Pat. No. 4,016,149
U.S. Pat. No. 4,332,717
U.S. Pat. No. 4,431,546
U.S. Pat. No. 4,623,625
U.S. Pat. No. 4,743,680
U.S. Pat. No. 4,771,128
U.S. Pat. No. 4,894,439
U.S. Pat. No. 4,908,434
U.S. Pat. No. 4,920,196
U.S. Pat. No. 5,252,216
U.S. Patent Publication No. 20070015169
Baird et al., FEBS Letter, Vol. 70 (1976) page 61
Fausnaugh, J. L. and F. E. Regnier, J. Chromatog. 359:131-146 (1986)
Harlow et al., Antibodies: Laboratory Manual, Cold Spring Harbor Laboratory Press: 511-552 (1988)
Kroviaski et al., J. Chromatography 449:403-412 (1988)
Marko et al., Anal. Bio., 121:382-387 (1987)
Miribel et al., J. Biochem. Biophys. Methods, 16:1-16 (1988)
Rikaken, Chemical Abstracts, vol. 102, No. 200711-u (1985)
Xuan et al., Chemical Abstracts, vol. 101, No. 187466a, (1984)

1. A column assembly comprising:
a body having an upper section, a reservoir section, and a lower section;
a reservoir formed in a reservoir section of the body and arranged to contain a binding matrix;
a top coupling member disposed adjacent the upper section, the top coupling member having a top passage in flow communication with the reservoir, and the top coupling member configured to couple to a syringe or a reservoir adapter;
an inner projection formed adjacent the lower section, the inner projection having a bottom passage in flow communication with the reservoir, and the inner projection sized and configured to connect to a vacuum manifold; and
an outer projection surrounding a portion of the inner projection, the outer projection sized to engage a centrifuge tube;
wherein the column assembly is adapted to couple, individually, to a syringe, a reservoir adapter, a vacuum manifold, and a centrifuge tube to enable fluid to pass through the binding matrix.

2. The column assembly according to claim 1, wherein the reservoir section, the inner projection, and the outer projection are cylindrical.

3. The column assembly according to claim 2, wherein an outer diameter of the outer projection is smaller than an outer diameter of the reservoir section.

4. The column assembly according to claim 2, wherein an outer diameter of the outer projection is between about 4 mm and about 11 mm.

5. The column assembly according to claim 1, wherein a bottom portion of the inner projection extends longitudinally beyond a bottom portion of the outer projection.

6. The column assembly according to claim 1, wherein the reservoir section, the inner projection, and the outer projection are integrally formed.

7. The column assembly according to claim 1, further comprising a support structure within the reservoir arranged to support the binding matrix.

8. The column assembly according to claim 7, wherein the support structure comprises a plurality of support ribs, the support ribs being integrally formed with a bottom portion of the reservoir.

9. The column assembly according to claim 1, wherein the top coupling member includes at least two mating tabs extending radially from a top portion of the top coupling member.

10. The column assembly according to claim 1, wherein the body comprises a thermoplastic polymer.

11. The column assembly according to claim 10, wherein the thermoplastic polymer comprises polypropylene, polystyrene, or a mixture of polypropylene and polystyrene.

12. The column assembly according to claim 1, wherein the top coupling member is ultrasonically welded to a top portion of the reservoir section.

13-25. (canceled)

26. A method for separating a compound from impurities comprising:
(i) loading a sample that comprises a compound and impurities onto a column of claim 1;
(ii) incubating the column under conditions wherein the compound binds to the column matrix;
(iii) removing impurities from the column under conditions wherein the compound remains bound to the column matrix.

27. The method of claim 26, wherein the compound is a nucleic acid.

28. The method of claim 27, wherein the nucleic acid is DNA.

29. The method of claim 26, wherein the sample comprises a cell lysate that contains the compound.

30. The method of claim 29, wherein the cell lysate is a bacterial cell lysate.

31. The method of claim 26, further comprising the step of (iv) removing the compound from the column.

32. The method of claim 31, wherein removing the compound from the column comprises loading an elution buffer onto the column under conditions in which the compound releases from the matrix and collecting the elution buffer comprising the compound.

33. The method of claim 32, wherein collecting the elution buffer comprising the compound comprises at least one procedure selected from the group consisting of spinning the column in centrifuge and applying a positive pressure to the top of the column.

34. The method of claim 33, wherein applying a positive pressure to the top of the column comprises applying the positive pressure with a syringe.

35. The method of claim 26, wherein removing the impurities from the column in step (iii) comprises at least one procedure selected from the group consisting of spinning the column in centrifuge, applying a positive pressure to the top of the column and applying a negative pressure to the bottom of the column.

36. The method of claim 35, wherein applying a positive pressure to the top of the column comprises applying the positive pressure with a syringe.

37. The method of claim 35, wherein applying a negative pressure to the bottom of the column comprises applying the negative pressure with a vacuum manifold.

38. The method of claim 26, wherein removing the impurities from the column in step (iii) further comprises washing the column matrix one or more times with a wash buffer under conditions wherein the compound remains bound to the column matrix.

39. The method of claim 38, wherein the wash buffer is removed using at least one procedure selected from the group consisting of spinning the column in centrifuge, applying a positive pressure to the top of the column and applying a negative pressure to the bottom of the column.

40. The method of claim 26, wherein the loading (i) and incubating (ii) are performed simultaneously under conditions wherein the compound binds to the column matrix.

41. The method of claim 26, wherein the method does not employ a centrifuge.

42. The method of claim 26, wherein the column matrix is silica.

43. A method for separating a compound from impurities comprising:
(i) loading a sample that comprises a compound and impurities onto a column of claim 1;
(ii) incubating the column under conditions wherein one or more impurities binds to the column matrix;
(iii) removing compound from the column under conditions wherein one or more impurities remain bound to the column matrix.

44. A purification kit comprising a column according to claim 1, and one or more additional components selected from the group consisting of: a preparative buffer, an elution buffer, a wash buffer, a syringe, a reservoir adapter, a centrifuge tube, a microcentrifuge tube, a collection tube, and a nuclease.

45. The kit of claim 44, wherein the kit further comprises an instruction manual for use of the kit.

46. The kit of claim 44, wherein the preparative buffer is a cell lysis buffer or neutralization buffer.

47. The kit of claim 44, wherein the reservoir adapter comprises a filter.
